(19) United States
(12) Patent Application Publication
(10) Pub. No.: US 2008/0140248 A1
(43) Pub. Date: Jun. 12, 2008

(54) Method and System for Determining Characteristics of Lumber Using End Scanning

(75) Inventor: Stuart G. Moore, North Vancouver (CA)

Correspondence Address: MCCARTER & ENGLISH LLP;CITYPLACE I, 185 ASYLUM STREET, HARTFORD, CT, 06103, US

(73) Assignee: STUART G. MOORE HOLDING INC., North Vancouver, BC (CA)

(21) Appl. No.: 11/665,935

(22) PCT Filed: Oct. 21, 2005
(86) PCT No.: PCT/CA05/01614

§ 371 Date: Apr. 20, 2007

(30) Foreign Application Priority Data
Oct. 21, 2004 (CA) 2,485,668

(51) Int. Cl. G06F 17/00; G06K 9/00; G01N 21/898; G01N 33/46

(52) U.S. Cl. 700/223; 382/141

(57) ABSTRACT

A process and equipment for an automated lumber end-scan system includes a conveyer to carry sawn lumber in a direction transverse to the axis of the boards, a light source to illuminate at least one butt end of each board as it passes by a scanning region, at least one digital camera to capture an image of each end face, and a processing unit to convert the digital signal into useable information. The digital signal is analyzed to obtain information about both natural and manufacturing defects that might be present at the end of the lumber and also to obtain further information about the properties of the lumber from the location of the pith, the growth rings and the grain pattern. This information may be used to augment the analysis of defects present in the entire board for determination of the final grade within an automatic lumber grading system. The system can also be used on a stand-alone basis and integrated into a non-automated grading area as a grader assist device.

### FIELD OF THE INVENTION

The invention relates to lumber processing methods and equipment, specifically methods and systems for determining the presence of lumber defects such as warp and cracks, as well as characterizing the quality of lumber by analyzing growth rings and locating the pith, using a scanning system.

### BACKGROUND OF THE INVENTION

In order to accurately grade a piece of lumber, the grader must be able to see all four sides of the lumber, and the two ends. As used in this specification, “sides” refers to the elongate side faces of a rectangular board and “ends” refers to the opposed end (butt) faces cut transverse to the grain to expose the growth rings. The term “lumber” means in general a sawn board, but it is contemplated that the invention may be adapted for use on whole logs or log segments.

In practice, a human grader is not able to effectively see the far end of each piece of lumber that passes by. The grader is able to glance at the far end of the piece if there is a mirror placed at the far side of the grading table. Given the maximum board length processed in a typical mill as being 24 feet, the mirror would normally be placed at a considerable distance from the grader. The shorter the board, the greater the distance the grader must look to see any defects in the far end of the piece. Additionally, a grader rarely looks at the near end of the piece unless he feels something wrong with the board as he manually turns it for examination. In mills that use automated board-turning systems the grader is able to glance at the near end of the piece since he does not have to stand physically close to the lumber as it passes by to manually turn it. Since the grader typically only has 2 seconds to view the entire piece of lumber and make a grade determination, the near-end and far-end information is never fully utilized, except in the obvious cases of the presence of end splits, rot or other gross defect. This assumes the table running at 30 pieces of lumber per minute. Many mills run at speeds in excess of this, or are capable of doing so. To abstract other information about a board, precise and elaborate calculations are required.

Automated lumber-grading systems have been developed which automate at least some of the grading process. For example, U.S. Pat. No. 5,412,220 to Moore discloses a system for conveying lumber in a transverse position across a grading table, with a bank of scanners positioned above the table for scanning exposed side faces of the boards as they are conveyed. Preferably, a board turner rotates each board, such that a second bank of scanners may then scan the opposed, previously hidden, board faces. The information derived from the scanners, such as the presence of knots, cracks, etc. in the board side faces is processed by a central processing unit, which in turn may transmit information to a trimmer to trim each board to an economically optimal length. While this system provides valuable information on an automated basis, other useful properties of the lumber are not readily assessed or extracted from such a system.

Automated grading of lumber or logs is also disclosed in American U.S. Pat. Nos. 5,023,805 to Aune et al.; 5,394,342 to Poon and 6,366,351 to Ethler et al.

The end faces of a board reveal information valuable to determining the characteristics of the board as well as its optimal trim. In particular, the end faces often display the tree growth rings which as described below provide a significant source of valuable information relating to characteristics of the board. As well, end faces can often show the presence and extent of board warp, splitting and wane. The growth rings can indicate the original location of the board within the tree, namely whether the board was cut from wood close to the pith or distant therefrom and the rate of growth of the tree. Higher value dimension lumber typically originates from trees that are more slowly growing, namely with closely-spaced growth rings, and closer to the centre of the tree. Proximity to the pith minimizes the size of knots and the extent to which any knots that are present are through knots. Other valuable information that may be obtained from viewing the end faces is the proportion of each board that is derived from heartwood, which is harder and more valuable, and that which is derived from sapwood, which is less valuable.

One particular aspect of lumber is its “wane”. Wane is defined as bark or lack of wood from any cause on the edge or corner of a piece of lumber. It naturally occurs in lumber sawn from the outer edges of the tree, i.e., close to the bark, although man made wane can occur on any piece of lumber. Thus, naturally occurring wane will always be on the barkside of the piece.

### SUMMARY OF THE INVENTION

In one aspect, the invention comprises a system for determining characteristics of lumber on an automated or semi-automated basis. The system is adapted to make calculations for each board respecting some or all of the tree's rate of growth, the nature of the wood grain, the angle of growth rings, along with the detection of end splits, pith and warp, all in real-time as the lumber is being processed. This information is abstracted and used as supplementary data in the detection and classification of knots and in making end-trim, cut-in-two decisions, and the determination of the final grade of a piece.

The system includes an illuminator to illuminate at least one butt end of each board, and preferably at least two illuminators, to illuminate opposing butt ends. The illumination source or sources may comprise ambient light but preferably illuminators such as high intensity LEDs or the like. The system further includes at least one digital image capture device such as a digital camera, to capture individual digital images of the at least one butt end of the board; a proximity sensor operatively connected to the digital image capture device, to trigger capture of images of the butt end of the board; a user interface for control of the system; and a signalling processing subsystem operatively connected to the image capture device and user interface. The signal processing subsystem is programmed to determine information regarding individual boards, based on digitized images of the board. This information is selected from at least one of the following:

the rate of growth of the lumber as determined from the growth rings;

the percentage of heartwood present in a piece in species where heartwood has a prominent color difference from sapwood;

the presence of heart and/or sap stain in the respective end of the board;

the presence and location of end splits;

the grain patterns;

the presence of warp (twist, bow, crook, and cup);

location of the pith (if present), and the approximate location of the pith when it is located outside of the piece;

the presence of heart center decay. Heart center decay is a localized rot that develops along the pith in certain species such as southern pine; and

the presence and extent of machine bite.

Preferably, the system includes a board conveyor which aligns a first end of the board, for image capture by a fixed-position image capture device. In order to accommodate variable-length boards, and opposed section image capture device may be moveably mounted on the opposing side of the board conveyor, linked to a proximity sensor for fore and aft movement to maintain a fixed distance with each successive board. Alternatively, the opposing image capture device may be manually moved to maintain the fixed position. In a further alternative, a plurality of opposing image capture devices may be provided to accommodate standard length boards with the length of the board determining which capture device is triggered.

The signal processing subsystem is programmed to extract information from the digitized butt end images, by a program which follows the flow charts described in FIGS. 10 and 11 of this patent specification.

### BRIEF DESCRIPTION OF THE DRAWINGS

FIG. 1 is a schematic diagram of the signal processing system of the lumbar scanners described herein;

FIG. 2 is a schematic diagram illustrating the operation of the device, wherein passage of a board triggers an image transfer, followed by a processing window for signal processing;

FIG. 3 is a state diagram of the image processing subsystem state module;

FIG. 4 is an end scan image showing growth rings and end split of a board;

FIG. 5 is a screen capture showing detection of a cup within a board;

FIG. 6 is a screen capture showing end split detection;

FIG. 7 is a digitized image captured by the camera, showing boards within the pith is located outside the piece of lumber under inspection;

FIG. 8 is a schematic diagram showing a piece of lumber in relation to its pith location, and the direction of scanning by the device;

FIG. 9 is an end scan image showing pith located inside a piece of lumber under inspection;

FIG. 10 is a flow chart of the image processing carried out within the central signal processor (second layer);

FIG. 11 is a flow chart showing the signal processing steps for detecting pith location and ring density measurement;

FIG. 12 is a side elevational view of a system according to the present invention;

FIG. 13 is a top plan view of the system;

FIG. 14 is a perspective view of a further aspect of the invention namely a wane-up board rotating system;

FIG. 15 is an image of a piece of lumber wherein the point of intersection of the lines is the calculated location of the pith, indicating a board that may be rotated to face downwardly by the wane-up system; and

FIG. 16 is an image of a piece of lumber showing a piece of lumber with the pith lying inside the board beneath the surface of the board and thus not requiring rotation.

### DETAILED DESCRIPTION

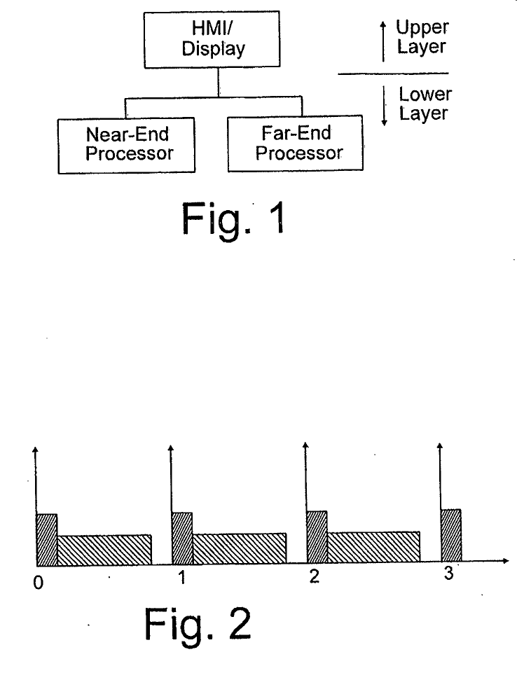

Referring to FIGS. 1 and 2, which illustrate respectively side and top plan views of a grading system and method according to the invention, the system 10 comprises a lumber conveyor 12 which transports lumber in a transverse position, that is, such that the elongate axis of each board 14 is oriented transverse to the direction of travel along the grading table conveyor system 12. The conveyor 12 transports lumber 14 across a grading table. The conveyor 12 comprises a plurality of spaced apart moving belts or chains 18 for supporting and conveying the lumber across the grading table. An example of a suitable conveyor system is described in U.S. patent application Ser. No. 5,412,220, which is incorporated herein by reference. In a typical sawmill operation, lumber 14 is placed on the conveyor 12 in an even-ended orientation, that is, with a first end of all boards being substantially aligned, while the opposed second end will vary in position depending on the board length. The table includes evenly spaced chain drive lugs such as Shark Fin™ lugs for transporting boards. Optionally, the system includes a pop-up board turner subsystem for rotating boards about their long axis in response to detection of wane within an individual board. The rotator comprises a rotateable pinwheel-shaped structure such as a Shark Fin™ rotator (described in U.S. Pat. No. 5,482,140 to Moore). The rotator includes a means to permit it to pop up for operation when a signal is received indicating a threshold level of wane in a board, for rotation of that board to carry out a multi-sided side scan

The system includes at least two digital cameras 20(a) and (b) or other image acquisition devices, mounted in generally opposed positions on either side of the grading table. The cameras 20 are positioned within a “scanning region” 22 of the grading table 12. As described below, the cameras 20 are each part of an image acquisition system. The lens 24 of each camera 20 is positioned to capture an image of each end of the board. Preferably, each camera lens 24 is substantially aligned with the horizontal axis of the boards, such that the image-capturing plane of the camera is parallel to the end face of the board. Preferably, the two cameras 20 are directly opposite to each other, although it is contemplated that the cameras 20 may be staggered in relation to each other. The cameras are mounted by mounting brackets 26, which in turn may be attached either to the grading table 12 or alternatively mounted to another structure in a suitable position. They are mounted slightly above the level of the lumber to avoid contact with the lumber. The mounts 26 should be sufficiently sturdy to minimize vibration and other unwanted movement of the cameras 20. A first camera 20(a) is mounted at a first side of the grading table 12 and is fixed in position relative to the grading table. This first camera 20(a) is positioned such that the lens is about 1 ft from the expected even-sided edges of the boards being conveyed along the table. The opposed second camera 20(b) is also mounted in a fixed position relative to the table 12. If the table is adapted for grading lumber only of a single length, only a single camera 20(b) is provided which is mounted such that its lens is also about 1 ft. from the expected position of the lumber edge as the lumber passes in front of this camera. However, it is expected that the table will be for use with a plurality of lumber sizes in two foot increments (8, 10, 12 feet long, etc.). For this purpose, a plurality of “far side” cameras 20(b), 20(c) 20(d) etc. are provided at corresponding locations to capture images of the far end of the lumber. In each case the camera is positioned such that its lens is about one foot from the expected position of the far end of the boards as these pass in front of the camera. Each of these cameras is mounted above the plane of the lumber to avoid contact between lumber and cameras.

An alternative to the provision of a plurality of “far side” cameras at staggered positions, is a single “far side” camera 20(b) mounted for variable positioning to accommodate boards of different lengths. These lengths will typically vary in 1 ft increments, from 8 feet for studs to 24 feet for dimension lumber. The camera 20(b) is associated with a linear track system or other precision positioning device available on the market; Such a system, which is known per se for other applications, relies on a distance measuring device to measure the relative board length and a controller which repositions the camera for each board as the same is conveyed in front of the camera.

Regardless whether a single camera 20(b) is provided with a repositioning system, or a plurality of fixed position camera, It will be seen that the respective distances between the board end faces and the corresponding cameras should be substantially equal. Preferably, this distance is about 1 ft, but it is contemplated that a greater or lesser distance may be provided, depending on the camera optics and other system design parameters.

In order to illuminate the butt ends of the boards, it is preferable that the system further includes an array of illumination sources 52, preferably a bank of high intensity LED lights, although depending on the image capture devices employed, ambient light may serve this purpose. The wavelength emitted by the illumination sources will be described below. A bank of Luxeon Lumiled™ high Flux LEDs is employed to illuminate a 2×2 foot area. For heat dissipation the lights are mounted to an aluminium plate, with cooling being assisted by one or more heat sinks and cooling fans. Preferably, a separate light source 52 is associated with each camera 20 and may be mounted to the camera or adjacent thereto for illuminating the opposing ends of the boards as. they pass in front of the cameras. It will be seen that multiple illumination sources on either side may be employed to provide more even lighting.

Each camera is operatively connected with a signal processing unit. It may also be connected to an optional proximity sensor 62 to trigger an image capture. The system can be synchronized with the automated lumber grading system to which it is connected such as ALGIS™ by disabling the proximity sensor and sourcing the trigger from this host system. The camera, the signal processing unit, and the optical sensor constitute the image acquisition system. This will be described in detail below. Conventional cooling means (not shown) are provided in the housing of the signal processing unit.

The camera is an industrial grade mega-pixel digital camera. It can be either monochrome or color depending on the species of lumber to be inspected. For example, for some redwood species it might be desirable to use infrared illumination to bring out the details in the image. Since color cameras include an infrared filter, monochrome-specific cameras would be used to capture the IR spectra images.

The camera has an external trigger input to facilitate triggered acquisition of images. It has programmable shutter speeds, capable of sub-millisecond exposure times in order to capture boards passing by at a rapid rate, such as 200 boards per minute or more. Another requirement imposed by the high board speeds is that the image transfer between the camera and the host processor be very fast. This requires a high-speed connection between the camera and the processing unit. CamLink™, Firewire™, Firewire B™, and Gigabit Ethernet™ can all be used.

The signal processing from the digital camera is carried out by a two-tiered-computing system architecture, shown schematically in FIG. 1. The lower level comprises a plurality of processors each linked directly to a single camera and dedicated to analyzing the raw image and extracting the pith, growth ring density and any other information that may be desirably abstracted at this level. This layer is the image processing layer. The upper level comprises a single central processor which receives input from the multiple lower level processors and makes a decision about the quality of the lumber based on this data. It has supervisory privileges over the lower layer and interfaces with the host automated grading system if the system is used as an add-on to an existing grading system.

The lower layer processors preferably each comprise an embedded processor running a real time operating system (RTOS) to maintain deterministic and stable operation. This could be a general purpose digital signal processor (DSP) or an Intel (tm)-based machine. Rugged industrial personal computers (PCs) running a stable operating system (OS) can also be used. However, to ensure determinism, an RTOS or real-time extension (RTX) is recommended.

The lower layer processors must have the requisite interface to the camera. For instance, if a CamLink connection is to be used a CamLink card must be installed in this layer. Another interface (e.g. Ethernet™, Firewire™, Firewire B™, Gigabit Ethernet™) is required to facilitate communication with the higher layer.

The upper layer processor is preferably a PC running a stable OS with graphic display capabilities. It hosts a graphical interface that serves as the human machine interface (HMI). This can be developed in any software of choice, e.g., Java™, .NET™, Visual Basic™, C/C++™, etc. In the case of a stand alone machine, parameters such as board speed and lumber size are entered using this interface. For the add-on machine, the parameters are passed through a data link interface with the host automated grading machine. An industrial grade laptop computer or a rack-mount industrial PC with a display unit can be used for this layer. The upper layer is absent when the system is configured to provide “wane up” analysis described below.

The light sources 52 are arranged to provide an even illumination pattern to highlight the features of interest in the image. Thus for different species (and hence, shades) of lumber the light sources with different color temperatures are used. In addition, since the exposure times are very short, the light source 52 should provide a high intensity. A constant light source or a synchronized strobe lighting system may be provided. In general, if the mill will be processing a diverse species of lumber, the illumination of choice will be warm white light, at a color temperature of between 3200K and 5500K. Redwoods and species that have a dominant red component will require light in the 625 nm to 700 nm wavelength range.

Spatial intensity variation across the image must be bounded to within 5% to maintain detection accuracy in the image processing algorithms. Additionally, the light source should be durable enough to maintain an intensity level of within 10% of its initial value after 12 months.

In one aspect the lights may comprise interspersed cool and warm white LEDs to maintain uniform illumination and a reasonable color temperature.

The lighting is mounted on the face of the housing that encases the camera, proximity sensor and the lower layer of the processing unit.

The proximity sensors 62 associated with the cameras 20 each comprise an optical device that activates when an object enters its field of view. When the device activates, it generates a pulse. This pulse is fed into the external trigger input of the camera and causes the camera shutter to activate and capture an image. Since different cameras have different external trigger voltage requirements (TTL or analog), care must be taken to ensure that the sensor output is compatible with the camera external trigger voltage requirements.

The sensitivity of the sensor is correctly tuned to prevent false triggering. This is includes the viewing angle and distance to the object. For example, an object within the viewing angle, but at 2 ft away should not trigger a sensor tuned for an object distance of 1 ft. Likewise, an object at 1 ft away that lies outside the viewing angle should not trigger any acquisition.

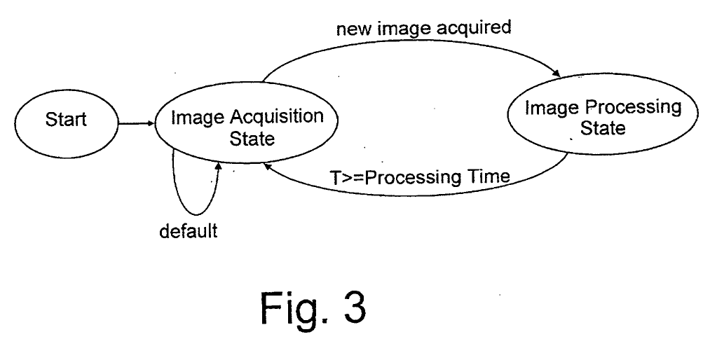

Further false triggering protection is built into the software design as depicted in FIG. 3. A detailed explanation of this follows in the next section.

### 3.0 System Operation

The system operates as follows: The system is first powered up. Then configuration information such as the size of the lumber, species, and scanning rate is entered through the HMI or communicated from the host automated grading system. This configures the system for the impending run. The program then enters an idle state, waiting for the trigger. The lumber conveyor 12 is then started. As a board enters the field of view of one of the proximity sensors 62, the sensor activates and sends a trigger pulse to the external trigger input of the camera. The camera 20 captures the image and sends it to the lower-level processor for analysis. Upon completion of the analysis, the lower level processor sends the results to the upper level processor for further analysis. The process repeats every time the proximity sensor is triggered. The analysis software is written such that it is able to complete the analysis before the arrival of the next trigger pulse. This sets a lower bound on the speed of the processor that can be used in the lower level module.

Spurious triggers are negated by disregarding triggers that occur within the processing window. 3.0 System Operation

The system operates as follows: The system is first powered up. Then configuration information such as the size of the lumber, species, and scanning rate is entered through the HMI or communicated from the host automated grading system. This configures the system for the impending run. The program then enters an idle state, waiting for the trigger. The lumber conveyor 12 is then started. As a board enters the field of view of one of the proximity sensors 62, the sensor activates and sends a trigger pulse to the external trigger input of the camera. The camera 20 captures the image and sends it to the lower-level processor for analysis. Upon completion of the analysis, the lower level processor sends the results to the upper level processor for further analysis. The process repeats every time the proximity sensor is triggered. The analysis software is written such that it is able to complete the analysis before the arrival of the next trigger pulse. This sets a lower bound on the speed of the processor that can be used in the lower level module.

Spurious triggers are negated by disregarding triggers that occur within the processing window. FIG. 2 illustrates graphically the time-slot allocations during each processing cycle. Once the camera gets a trigger signal, it transfers the acquired image to the signal processing unit during the “Image Transfer” slot. The system then enters the “signal processing window” during which all the image processing and image analysis tasks are undertaken. During this time, all triggers that occur are ignored. A guard time is included to make sure the image processing has sufficient time to complete before the arrival of the next trigger signal. This is accomplished by making the image processing tasks time-bounded, i.e., an upper bound is imposed on how long these processes can take to execute.

The image transfer time is a camera parameter determined by the speed of camera-processor interface and the pixel resolution of the camera. The processing window is set to the longest time it would take the system to analyze the image and report the data. It is determined by the speed of the processor and the size of the image to be processed. This window is empirically established during the code development stage by profiling the code as it executes. Profiling is a technical term that describes tracing the program as it runs to determine how much processor resources each sub-program uses. Here, “processor resources” refers to both CPU time and memory requirements. The next section briefly describes how profiling is used to set bounds on the duration of the various sequential activities in each time slot, as shown FIG. 2.

The program is started with the profiler enabled. After 20 or more runs, the program is stopped and the profiler output is analyzed. This data shows the average length of time the program takes to execute as well as the longest time it takes. Since the system has to accommodate the worst case scenario, the processing time is chosen to be longer than the longest time it took the program to execute.

A state machine is then designed with the following two states: Image Acquisition State and Image Processing State. In the Image Acquisition State, the program acquires a new image once it receives a trigger signal. This forces a transition to the Image Processing State. Once in this state, a timer is started. This timer counts down from a value equal to the processing window duration in FIG. 2. This is a background process. In the foreground, image processing routines execute. Once image processing is complete, the program waits for the expiry of the timer before transitioning back to the Image Acquisition State. In the event that the timer expires before image processing is complete, the image processing is stopped and a “processing incomplete” flag is set before the program can transition to the Image Acquisition State. This flag signals the higher layer that it will only be receiving partial results and that there was possibly a problem with the system. The state diagram of for the entire image processing subsystem is shown FIG. 3.

### 4.0 Image Processing Subsystem

### 4.1 Overview of the Image Processing Subsystem

The image processing subsystem resides on the individual processors connected to the image acquisition device. As previously stated, this subsystem runs image analysis algorithms on the acquired image. These algorithms do the following:
1. Calculate rate of growth and wood density from the growth rings slope of grain, honeycomb, and white speck;
2. Determine the percentage of heartwood present in a piece in species where heartwood has a prominent color difference from sapwood;
3. Detect the presence of heart and/or sap stain in the ends of the piece;
4. Find end splits;
5. Analyze grain patterns;
6. Detect and measure the presence of warp (twist, bow, crook, wane, and cup);
7. Locate the pith (if present), and the approximate location of the pith when it is located outside of the piece;
8. Detect the presence of heart center decay. Heart center decay is a localized rot that develops along the pith in certain species such as southern pine;
9. Determine and accurately measure machine bite. A depressed cut of the machine knives at the end of the piece; and
10. Find location of knots and determine a grade of knot, including determination of single, two, three, and four-faced knots.

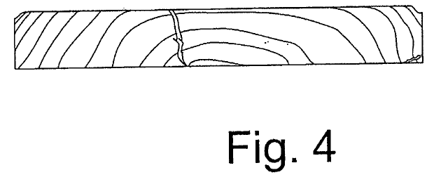

FIG. 4 is a photograph of a board end view showing typical growth rings and end splits.

### 4.2 Image Processing Sequence

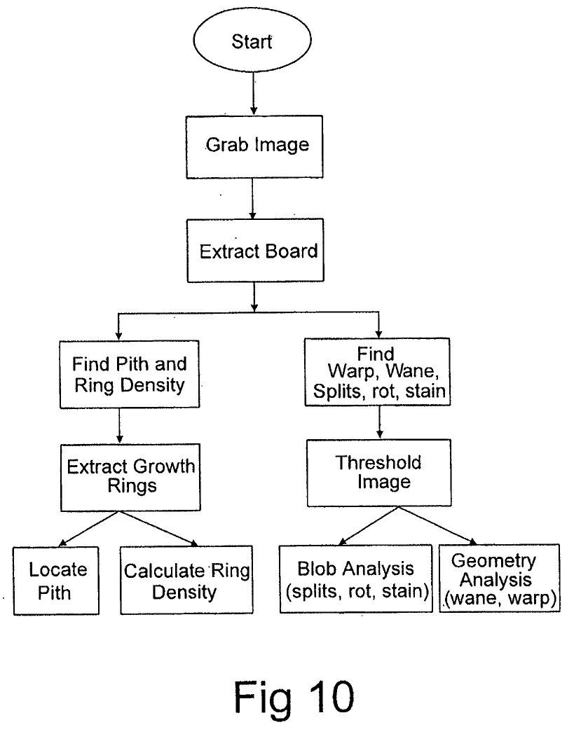

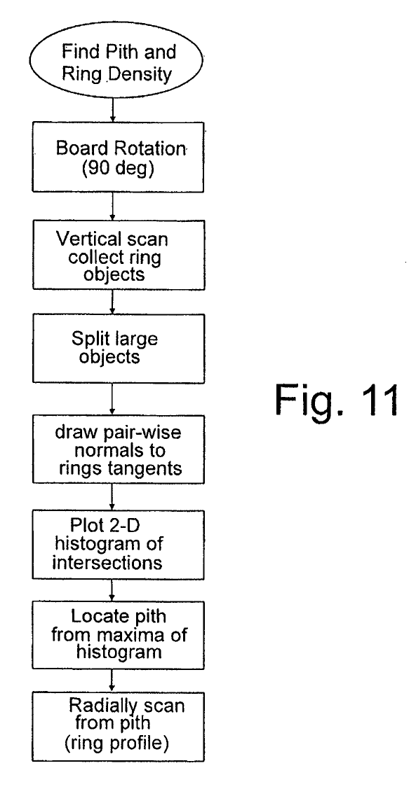

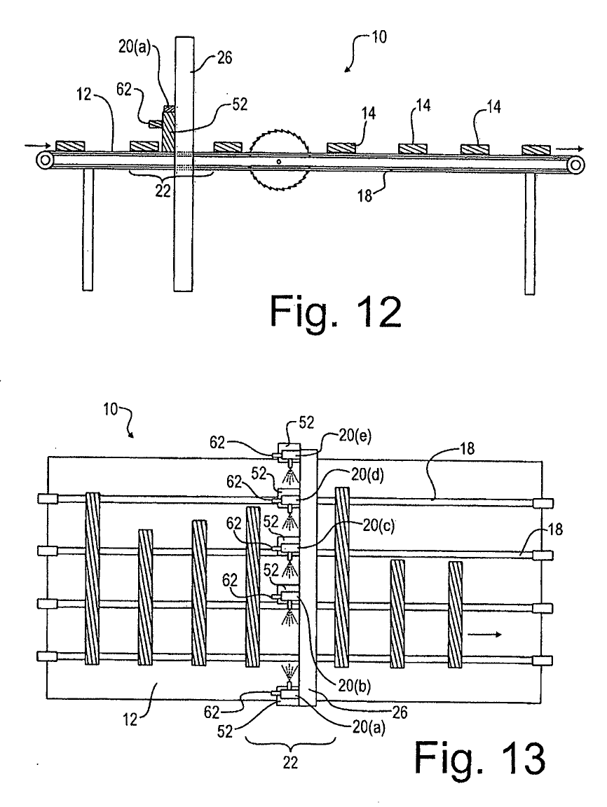

The following describes the sequence of steps in the image processing subsystem. Flowcharts have been provided in FIGS. 10 and 11. The first stage is board extraction. Here, simple thresholding algorithms are applied to the image to remove the background and retain the board area only. Then the sequence splits into two paths, as seen in FIG. 10.

### 4.2.1 Warp, Wane, Splits, Stain, and Rot Detection

The amount of twist, crook, and cup in the board can be calculated by measuring the displacement of the extracted board with respect to the horizontal plane. In other words, an analysis of the geometry of the extracted image is performed. The system is first calibrated with non-warped boards of all the various sizes and the calibration parameters are stored in the processor memory. Similarly, the amount of wane can also be determined by looking at the edges of the board. For example, FIG. 4 shows wane at the top right hand edge.

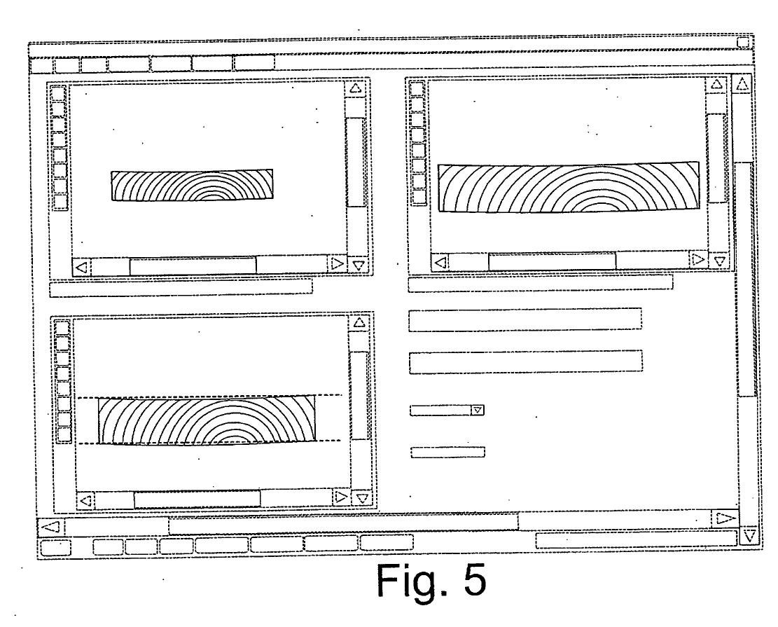

FIG. 5 shows a screen capture of cup detection. The original image is shown on the top left hand of the picture. Board extraction removes the background to yield the image on the right. Cup is measured by finding the maximum deviation from the horizontal line joining the two ends of the board, i.e., the deviation at the lowest point. This is indicated in the image in the bottom left half of this picture by a red perpendicular drawn from the horizontal line to the lowest point of the board.

Following board extraction, more sophisticated thresholding, color analysis, and blob analysis are done to extract other parameters.

Color analysis is done to detect the presence of heartwood or sapwood, as well as heart center rot. This analysis takes advantage of the reflectance and absorption properties of different shades of wood.

End splits are detected by simple thresholding of a monochrome image. This image could be grayscale or the result of extracting a single color component from an RGB image.

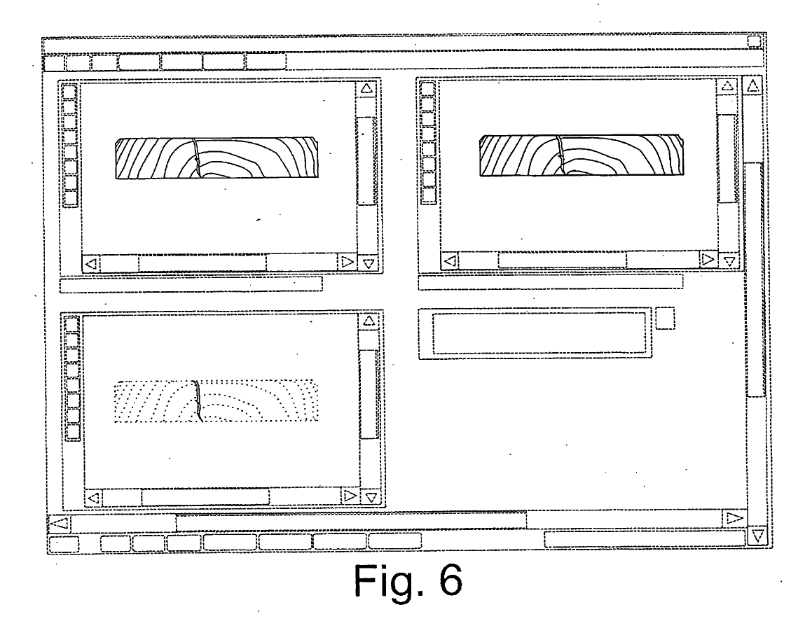

FIG. 6 shows a screen capture of the end-split detection process for the image in FIG. 4. The top left is the original image. The bottom left image is a binary image of the split itself. This is overlaid onto the original image in the image on the right.

### 4.2.2 Pith Detection and Average Rate of Growth Measurement

The determination of average rate of growth and location of the pith require more intricate processing, as can be seen FIG. 11. The first stage involves extracting the growth rings. This is a multi-step process premised on the following observation:

In temperate climate there are two distinctive growth seasons for a tree, leading to a banded structure on the cross section of a tree. The rapid growth spring season is characterized by a broad band while the slow growing summer season is characterized by a narrow band, marked by a darker shade than the spring band. Thus, theoretically, a contrast-based threshold can yield a binary image of the ring pattern, with the hits being the summer rings and the misses the spring rings. However, because of noise due to pitch and bad sawing, this method is not practicable. The following is done, instead:

Lines are drawn parallel to the narrow side of the board and a binary image is generated in which the hits correspond to the intersection of these lines with the summer rings. In FIG. 4 this would correspond to scanning the image column wise, from left to right, which would be very slow because of the way images (arrays) are stored in memory. Thus, the image is first rotated by 90° prior to scanning to speed up the process,

The resulting binary image will contain hits from true rings and false rings. Since every column is scanned, some connected pieces regions emerge in the binary image, some of which are clearly false because of pitch, dirty or uneven sawing. Therefore, to make the system more robust, large connected objects are split into smaller independent objects.

Consider the cross-section of a hypothetical tree with perfectly circular growth rings. All normals to tangents to growth rings would pass through the center (pith) of the tree. In an ideal tree with perfectly circular rings, all that is required is to find the point of intersection of two such distinct normals to locate the pith. However, since growth rings are not perfectly circular, and it is impossible to accurately extract the rings due to noise, the following procedure is used:
1. Identify candidate pairs of points lying on the same ring, and construct normals to tangents at those points. Multiple pairs are used for each ring to increase robustness.
2. Plot a 2-D histogram of the intersection of the normals, i.e., plot the locus of the x- and y-co-ordinates of the intersections.

The pith position is given by the intersection of lines passing through the peaks of the two histograms.

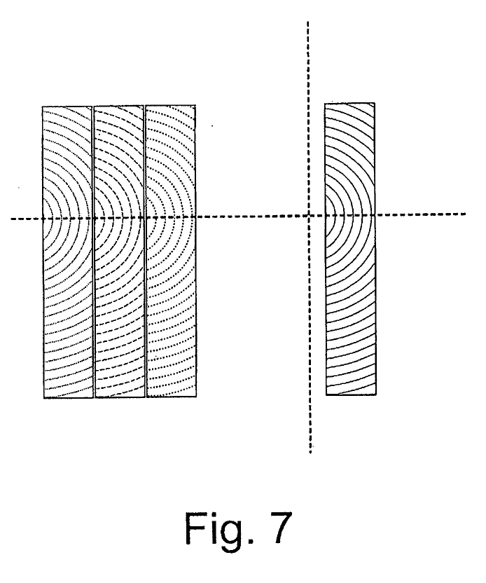

FIG. 7 is a series of end-scan images showing the pith located outside the piece of lumber under inspection. This piece is said to be free of heart center (F.O.H.C) or side cut.

To calculate the growth ring density, the following procedure is followed;
1. Starting from the pith, a radial scan of the ring image is done. At each position/orientation, the number of intersections of the scan line with candidate growth rings is recorded.

2. A histogram or profile of the intersections is plotted.

The peak of the histogram gives the average number of intersection, and hence the average ring density.

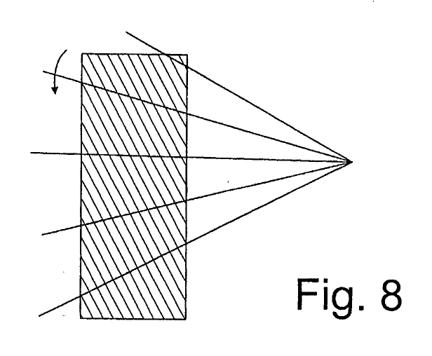

FIG. 8 is an illustration of radial scanning starting from the pith. The arrow shows the scan progression.

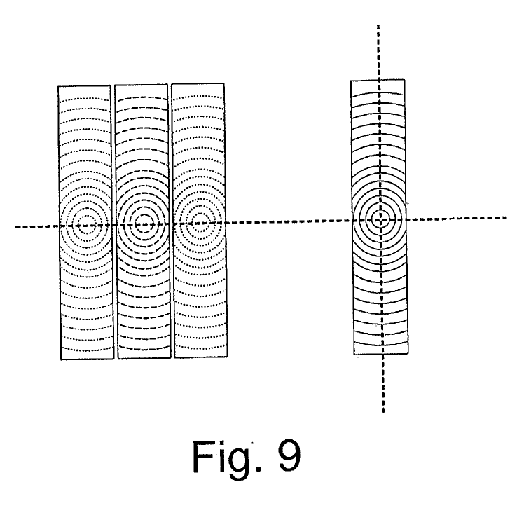

In FIGS. 8 and 9, looking from left to right, the first image is the original image. The second image shows the output of growth ring detection, after splitting the large objects (see FIG. 11). The third image is a reconstructed image, showing how the original image would have looked like if the growth rings had been evenly spaced. The fourth image is the original image underlain to show the exact distance of the pith position with respect to the board. The position of the underlay image is precisely calculated to give the exact pith location. The yellow dots are the candidate pith locations as determined by the pair-wise normals alluded to in the previous section. The histogram filters off all the spurious point, leaving one true pith position defined by the two maxima of the 2-dimensional histogram.

Even though the rings are hardly discernible in FIG. 7, the algorithm used to process the signal accurately detects the pith. The reason for this is that because of the splitting of the ring objects into smaller objects. What this does is effectively increase the number of valid ring-pairs. This leads to more hits at the correct pith position. The same can be said for FIG. 9 where the pith, seen as the dark X-like features in the original image, severely distorts the ring structure.

The average rate of growth is measured on a line at right angles to the rings in an area representative of the average growth in the cross section at either one end or the other. This line should be 3″ long, if size permits. And since our method already calculates the average ring density, the number of rings in a 3″ section of line can be found by simple multiplication.

In boxed heart (when the pith lies inside the piece of lumber under inspection), the average rate of growth is measured on a radial line starting at a quarter of the least dimension away from the pith. Since the co-ordinates of all the candidate rings are known, the intersections of the scan line with rings inside the excluded area are removed from the density score.

A stand alone pith detection system may be provided to incorporate within an existing lumber grading table. This system comprises a mounting stand, a housing for a CPU, digital camera and light source, a trigger system, and associate wiring.

In one aspect, the signal processing locates the center of the lumber heart, via the steps described above. In order to provide this, allowance must be made for dense versus non-dense lumber. An initial pre-processing stage is carried out to distinguish dense from non-dense lumber. Separate processing algorithms are used for each.

Finally, a thresholding program is employed to overcome interference which may occur with prominent saw-marks on the lumber ends.

FIG. 9 is an end scan showing pith located inside the piece of lumber under inspection. This is termed “boxed heart”.

### Knot Detection

Grading: The grading program includes grading for “combination knots” and unsound knots. When two or more face knots are located such that if a normal to the sides passes through all of them, they are said to be “in the same cross section”, and therefore should be graded as a “combination knot”. The size of this combination knot is the sum of the sizes of the individual knots, and this cannot exceed the maximum allowable centerline knot. If one of the knots is an edge knot, however, then if the grade due to the edge knot individually is lower than that due to the combination knot, the lower grade takes precedence.

### Wane Up Detection

The Wane-Up System detects pieces that are barkside down and signals the boardturner to turn them barkside up. This is accomplished by scanning one end of lumber and using growth-ring information to determine the orientation of the piece. Since the system does not detect wane, but rather, determines where wane would be, it is able to pick out pieces that have pencil wane. It can be deployed on the edger optimizer or the planer infeed.

The wane up detection system is comprised of two subsystems, namely, the detection subsystem and the boardturning subsystem. The whole system fits in about 5-6 feet. The detection subsystem relies on the components described above namely a megapixel industrial color digital camera, a trigger sensor, an LED lighting panel mounted at the front of the box that houses the subsystem, an industrial single board computer (SBC) processing unit running a real-time operating system (RTOS), and image analysis software running on the SBC. The trigger sensor signals the camera to acquire an image whenever it detects the presence of a board in front of the camera. The acquired image is transferred to the SBC which applies proprietary algorithms on the image to detect determine board orientation. If barkside down is detected, the SBC outputs a high signal to one of its digital input/output (IO) lines which tell the boardturning subsystem to turn the board over. The two subsystems must be correctly synchronized to ensure that the right boards are turned. The Wane-up system thus interfaces with the Sharkfin Boardturning System.

The Wane-Up box has three indicator lights mounted at the top of the box: Green, Red, and Amber. The amber light flashes each time a board that needs to be turned is detected. The green light signifies “ready” or correct operation, while the red light indicates system failure or program stoppage for any reason. The red button to the left of the indicator light in Figure below is the power switch to the module.

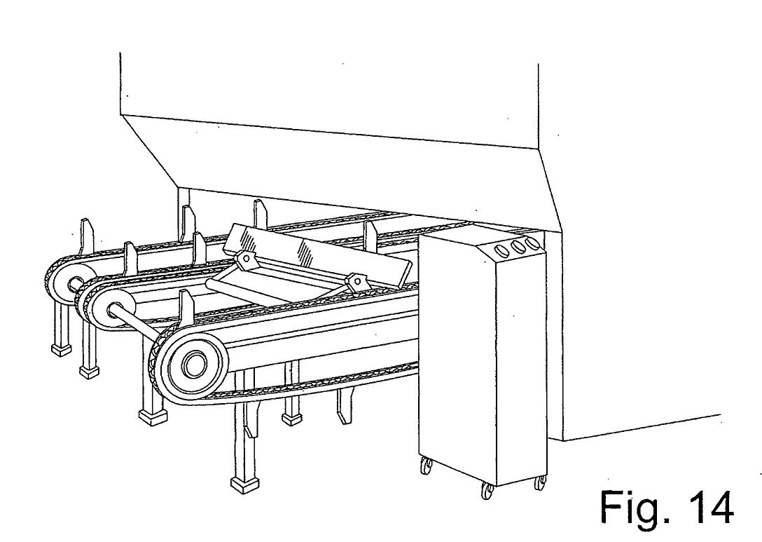

FIG. 14 shows the wane-up system with board being turned into the “wane-up” position by the board turner.

Lighting comprises long life LEDs and is designed to provide both even illumination as well as the a color temperature to enhance the defect detection process.

The system has been implemented using a real-time operating system (RTOS) which ensures reliable, deterministic operation. Thus, the system is not prone system hang-ups that plague other non real-time desktop operating systems.

The single board computers use solid state compact flash drives instead of hard drives to avoid having moving parts in the systems. Hard drives quit when the motor burns out. With compact flash drives those problems are eliminated. The compact flash drive is small, light and durable, and thus improves system reliability.

### System Operation

At power on, the system goes through a series of self-checking and calibration procedures. It tests proper camera connection and/or operation, trigger connection and functionality, and indicator lights. When all the tests pass, the three indicator lights flash in sequence once, and then the green light comes on, signalling to the mill that the Wane-Up is now ready for operation. If any of the subsystems should not test out, the red light comes on indicating system failure. This error condition is entered into the error log stored on the compact flash drive on the single board computer and indicates precisely where and when this error occurred. Algis engineers analyze the frequency and scenario of these error conditions to further improve system robustness.

The system has been designed to be as self-healing as possible. When an error condition occurs, the system tries to recover from it by rebooting itself.

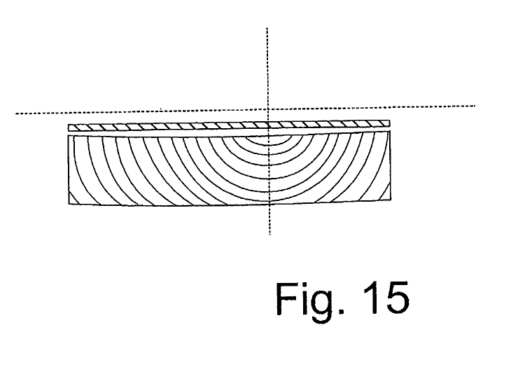

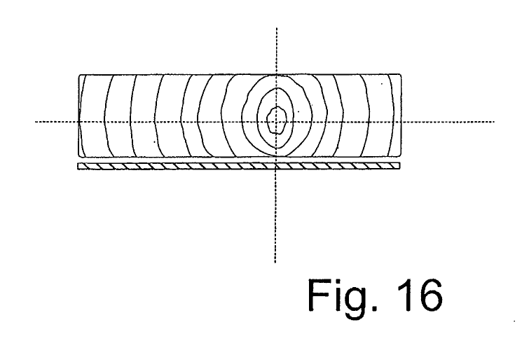

FIG. 15 shows a piece of lumber on a table moving from right to left. The green bar shows that the pith lies above the surface of the board. FIG. 15 shows a free of heart center (F.O.H.C) piece of lumber with sapwood side resting on the table. The intersection of the blue lines shows the extrapolated pith position. In this example, the piece needs to be turned over. In FIG. 16, a piece of lumber with boxed heart is shown. The green bar shows that the pith lies below the surface of the board. The pith lies inside the piece, i.e., the piece is cut from the center of the tree and therefore will not have natural wane. Thus this piece will not be turned.

The green bar plays the role of the amber light on the Wane-Up box. When the green bar is above the board, the amber light is ON, and vice versa.

### System Requirements

Since correct operation of the Wane-Up is premised on the detection of growth rings, precision end trimming (PET) must be provided by the mill. The lumber must be even-ended as the camera-board distance must stay constant. In order to satisfy small footprint requirements, the front of the lens is about 1 ft from the lumber line. And since the camera is mounted inside the box, the front of the box can be as close as 6 inches from the lumber line.

It is recommended that the Wane-Up system be operated with the Sharkfin Boardturning System™.

### 4.3 Data Aggregation

The data from the two end scans is combined at the upper level to determine the quality of the piece of lumber. An interface is defined, a priori, specifying how the data is to be passed to the higher layer. This is specified down to the exact number of bytes for each defect reported. Special delimiters are used to indicate the end of one defect and the beginning of another. The higher layer verifies correct reception of the report from the lower layer by counting the bytes received as this is always constant and predefined. The data reporting takes place every clock cycle, at the end of the processing window (See FIG. 2).

Some measure of grading takes place at this level. However, this grading is only partial and can only be used as supplementary information. The next few sections take a detailed look at how data for a specific feature is treated, beginning with growth rings.

Growth ring density information gives an indication of the strength of the piece of lumber. The denser the growth rings pattern, the stronger the piece. The lumber is classified as “dense” if it satisfies a minimum threshold for growth rings per inch. Since this need only be done for either the near-end or the far-end, the system has redundancy to ensure more accurate measurements.

Presence or absence of pith indicates the quality of knots in the piece of lumber. Since the pith is the center of the tree and knots (branches) grow from the center, outwards, the presence of the pith in a piece of lumber indicates that the knots are not “through knots”, i.e., co-located knots on opposite faces of the lumber are distinct. On the other hand, if pith is not present in a piece of lumber, knots appearing on one face will go through the piece to the other face. The direction of the pith is important in the calculation of knot sizes. The size of the knot is always smaller in the direction of the pith for a through knot.

The amount of warp (cup, crook, twist and bow) detected is compared against the warp thresholds for the various grades to determine the highest grade for the piece of lumber under inspection. Whereas cup can be detected based on one end scanner or the other; twist, crook, and bow require a comparison of dimensions measured at each end.

The presence of end splits on one or both ends is also indicated. This is used to make trimming decisions downstream. For example, if a piece of lumber is clear, except for end splits at one end, the mill operator can set the saws to trim off 2 ft from the side with the end split. The resulting piece goes into a higher grade and fetches a higher price.

All this data is put into a data structure and reported to the host automated grading system every clock cycle. When the ALEVS is running in a test or diagnostic mode, this data is also written to an output file for analysis.

Although an embodiment of this invention has been described in detail, the scope of the invention is not limited in any respect by this description. Rather, the full scope is set forth in this patent specification as a whole including (but not limited to) the accompanying claims. The invention also includes all functional equivalents to elements set forth in this specification which have not been explicitly limited in scope.

1. A system for grading lumber boards while said boards are being conveyed in a direction transverse to the board axis, comprising:
a first digital image capture device to capture individual digital images of at least one butt end of said boards;
a proximity sensor operatively connected to said digital image capture device to trigger said individual image capture;
a user interface; and
a signal processing subsystem operatively connected with said digital image capture device and user interface, said subsystem for calculating information in respect of individual boards from said individual digital images and conveying at said information to said user interface, said information being selected from at least one of the following:
the rate of growth of the lumber as determined from the growth rings;
location of the pith (if present), and the approximate location of the pith when it is located outside of the piece.

2. A system as defined in claim 1, wherein said signal processing subsystem further determines one or more of the following from said digital images:
the percentage of heartwood present in a piece in species where heartwood has a prominent color difference from sapwood;
the presence of heart and/or sap stain in the respective end of the board;
the presence and location of end splits;
the grain patterns;
the presence of warp (twist, bow, crook, and cup);
the presence of heart center decay; and
the presence and extent of machine bite.

3. A system as defined in claim 1, further comprising a second digital image capture device positioned to capture individual digital images of opposed ends of said boards, said second digital image capture device being operatively connected to said proximity sensor and said signal processing subsystem.

4. A system as defined in claim 1, further comprising at least one illumination source for illuminating butt ends of the boards at the time of capture of said digital images, selected from a constant illumination source or a strobe operatively connected to said proximity sensor.

5. A system as defined in claim 3, wherein said illumination source provides illumination at a frequency range selected according to the wood species, said frequency range comprising between 625 and 700 nm for species with predominant red coloring and at a color temperature of between 3200K and 5500 K for diverse species.

6. A system as defined in claim 1, wherein said signal processor carries out the following sequence of steps:
receiving digitized image of board butt end;
extracting location, position, and configuration of features of board butt end represented by color or shade differential, said features comprising rings, cracks, warp of board, wane, splits, rot, and staining;
from said ring information, determining a pith location and finding ring density;
from said pith and ring density information, extracting growth ring information and thereby locating pith location and calculating ring density;
from said warp, wane, split, rot, and stain information, obtaining a threshold image of said butt end;
from threshold image, conducting a blob analysis to determine split, rot, and stain;
from threshold image, also conducting a geometry analysis to determine wane and warp of said board; and
transmitting said information in the form of data input into a data structure and reported to a host automatic grading system.

7. A system as defined in claim 6, wherein said steps of processing information relating to pith location and ring density comprise the steps of:
rotating said boards by substantially 90°;
conducting a vertical scan of said boards and collecting ring objects;
splitting large objects detected on said board butt ends;
determining an array of lines which are normal to the tangents of said rings detected by said scanners;
plotting a two-dimensional histogram of intersections of said lines;
locating the pith of said board from the maxima of said histogram; and
radially scanning from said pith to determine a ring profile, the peak of density profile comprising the average ring density.

8. A system as defined in claim 1, further comprising means for rotating said boards about their elongate axis by approximately 90° or more prior to a digital image capture of said butt ends of said board, for conducing a vertical scan of said boards.

9. A system as defined in claim 1, further comprising a lumber conveyor for conveying board parts said at least one image capture device.

10. A system as defined in claim 2, wherein said second digital image capture device is mounted to a repositioning device for maintaining a generally constant spacing between said device and the corresponding end of said individual boards.

11. A system as defined in claim 2, further comprising a plurality of said second image capture devices mounted in a plurality of fixed positions above the plane of said lumber.

12. A method of grading lumber comprising the steps of providing a system as defined in claim 1, determining with said signal processing subsystem any of the variables defined in claim 1 and assigning a grade to said boards in accordance with said information.

13. A method as defined in claim 12, further comprising the step of transmitting said information to a board cutter for trimming said board in response to said information to achieve an economically optimum trim thereof.

14. A system for grading lumber boards while said boards are being conveyed in a direction transverse to the board axis, comprising:
a first digital image capture device to capture individual digital images of at least one butt end of said boards;
a proximity sensor operatively connected to said digital image capture device to trigger said individual image capture;
a user interface;
a board rotator for selectively rotating boards on said conveyor in response to a signal received by said rotator
a signal processing subsystem operatively connected with said digital image capture device, board rotator and user interface, said subsystem for calculating wane direction information in respect of individual boards from said individual digital images and conveying at said information to said user interface and said board rotator for selectively rotating said boards to maintain a wane-up position of said boards on said conveyor.
